(19) United States
(12) Patent Application Publication
(10) Pub. No.: US 2005/0065526 A1
(43) Pub. Date: Mar. 24, 2005

(54) Fixing device and applicator therefor

(76) Inventors: Tim Drew, Dundee (GB); Paul Allcock, Dundee (GB); David Rowley, Dundee (GB)

Correspondence Address: GIFFORD, KRASS, GROH, SPRINKLE;ANDERSON & CITKOWSKI, PC, 280 N OLD WOODARD AVE, SUITE 400, BIRMINGHAM, MI, 48009, US

(21) Appl. No.: 10/497,936

(22) PCT Filed: Dec. 4, 2002
(86) PCT No.: PCT/GB02/05477

(30) Foreign Application Priority Data
Dec. 4, 2001 (GB) 0128953.7

(51) Int. Cl. A61B 17/58

(52) U.S. Cl. 606/72

(57) ABSTRACT

A fixing device is disclosed which is primarily for use in orthopaedics. In one disclosed embodiment the fixing device includes an elongate body having a throughbore, wherein the elongate body has a number of spiral slots in the surface thereof. In use, the fixing device is inserted into an aperture in a bone and a screw is tightened within the throughbore of the elongate body, causing the body to expand and the slots to be closed in order to secure the fixing device in place within the aperture in the bone. An applicator device is also disclosed which in use holds the fixing device in place when a screw is tightened therein.

The present invention relates to a fixing device, and in particular, but not exclusively, to a fixing device for use in orthopaedics. The present invention also relates to an applicator device for use with the fixing device.

It has become common practice in orthopaedics to use devices which can be fixed to or within bone structures, normally damaged or weakened bone structures, to provide artificial support. For example, certain bone fractures, such as those in osteoporotic (soft or weak) long bones may require the use of an intramedullary device to be fixed within the damaged bone to provide support while allowing the bone to heal. However, the use of intramedullary nails is extremely restricted in some circumstances, such as near joints or in the presence of prosthesis, and in these cases it is more appropriate to use an onlay device.

Onlay devices are normally fixed to the surface of a damaged or weakened bone structure by the use of adapted screws, such as cancellous bone screws, which are driven directly into the bone. If such screws are over tightened, however, a weak fixation results as the internal thread form is stripped from the bone. Under these circumstances there are limited means at the disposal of a surgeon to rescue the situation, none of which are entirely satisfactory.

Such rescue means include the use of a cable plate which potentially devascularises the bone by circumferential dissection and relies upon a large and expensive implant being kept on the shelf and only used occasionally. Additionally, it is known to use a polymethylmethacrylate (PMMA) bone cement inside the medullary cavity. This provides a secure fixation but can make the revision of the fixation difficult if it fails.

It is therefore an object of the present invention to obviate, or at least mitigate, one of the aforementioned disadvantages.

According to a first aspect of the present invention there is provided a fixing device for use in orthopaedics, said fixing device comprising an elongate body portion having a longitudinal bore extending therethrough, said bore adapted for receiving a threaded body, wherein, in use, said fixing device is inserted into an aperture in a bone structure and a threaded body is inserted into the bore and tightened therein by rotation relative to the fixing device, causing axial compression of the fixing device and circumferential expansion of at least a portion thereof to secure the fixing device within the aperture of the bone structure.

Thus, a screw or the like may be tightened within an existing aperture in a bone in which a screw may have previously been over tightened destroying the internal thread, or alternatively where the bone is too weak to support solely a screw fixation. This, therefore, is particularly advantageous when a device, such as an onlay device, is required to be fixed to the surface of a bone. Additionally, when an onlay device, for example, is to be replaced, the fixing device may be inserted into the existing apertures such that strong fixation results, without the requirement for re-using screws or for using larger diameter screws to re-tap a thread causing localised weakening of the bone at a possible vulnerable point.

Additionally, the device may be used in an aperture formed by drilling into the cortical wall of a bone structure, and as such is not limited for use in an existing aperture, formed by a cancellous screw, for example.

Preferably, the fixing device further comprises a head portion having a diameter greater than that of the body portion and selected to be greater than that of the bone aperture into which the device is to be inserted. This, therefore, prevents over insertion of the device into a bone structure.

Advantageously, the device may be used in existing screw holes to eliminate the requirement for excessive enlargement of such holes which may further compromise an already weakened structure. This also allows the fixture device to be effectively used in rescue situations where, for example a fixture screw or a bone fixture is to be replaced.

Conveniently, the fixing device may accommodate standard cancellous bone screws, thus allowing existing screws to be used without excessive stock requirements of specialised screws.

Preferably, the device is of a sufficient length so as to extend through the cortical wall of a bone and into its intramedullary cavity. Preferably, the portion of the fixing device which extends into the cavity will expand to form a load bearing surface on the inner surface of the cortical wall, providing increased strength and resistance to adverse loading and pull out or extraction forces.

Preferably, the length of the device does not exceed the diameter of the intramedullary cavity of the bone plus the bone cortical wall thickness.

Conveniently, the wall thickness of the body portion of the fixing device varies over the length thereof such that the body portion adjacent the head portion has a thinner wall thickness than that of the remaining body portion. Preferably, the mid wall section of the body portion has an increased wall thickness profile relative to the remainder of the body portion.

Preferably, in use, when a threaded body is tightened within a fixing device located in a bone aperture, the axial forces produced will initially collapse and buckle the thin walled portion of the fixture device, causing circumferential expansion and engagement with the inner surface of the aperture. Such initial expansion accommodates for various aperture diameters and cortical wall thicknesses, as the thin walled portion allows for significant axial shrinkage of the fixing device.

Conveniently, further tightening of the threaded body will cause circumferential expansion of the mid wall section of the body portion located within the intramedullary cavity, producing a distended mid wall section. At this stage, the distended portion prevents the fixing device from being extracted from the aperture under any significant extraction force.

Preferably, continued tightening of the threaded body will cause further axial compression of the fixing device and will cause the expanded mid section to be compressed against the inner face of the cortical wall, firmly securing the fixing device within the bone structure.

Preferably, the fixing device further comprises at least one slot located in the body portion thereof. More preferably, the fixing device comprises a plurality of slots located in a mid section of the body portion. The provision of slots provides a localised weakness in the fixing device to ensure circumferential expansion will occur at the required location and in the required manner to provide proper expansion and fixation.

The slots may be, for example, longitudinally extending slots aligned parallel to the longitudinal axis of the fixing device. Alternatively, the slots may be tear shaped apertures. In a preferred embodiment of the present invention, the slots are spiral apertures. Preferably, the spiral apertures wind in a clockwise direction; that is, in the same direction as a right-hand screw thread. Alternatively, the spiral apertures may wind in an anti-clockwise direction.

Providing spiral apertures allows for a robust fixation as upon tightening a threaded body within the throughbore the axial forces will cause the slots to close producing a localised radial expansion of the body portion which provides a solid structure on the inner face of the cortical wall. This renders the fixing plug, in use, far stiffer and more resistant to bending then those fixing plugs which comprise slots which remain open or are indeed widened when the plugs are tightened in place.

Preferably, each spiral aperture extends at least 45°, and more preferably at least 90° around the circumference of the fixing device.

Preferably also, each spiral aperture extends less than 360° around the circumference of the fixing device.

It would be obvious to a person of skill in the art, however, that the spiral slots may extend around the circumference of the fixing device to any extent which would provide sufficient expansion and strength of the fixing device when in use.

The fixture device may be manufactured from a synthetic fibre material such as nylon, for example. Alternatively, the fixing device may be made from a plastics material such as polytetrafluoroethylene (PTFE). In general, any material may be used which is biocompatible with living tissue, and which will not degrade once fixed in location.

Preferably, the fixing device is sterile and provided in a hermetic packaging to prevent contamination until such time as the device is required.

In one embodiment of the present invention, the fixing device has a length of between 20 and 25 mm, preferably, 23 mm, an outer diameter of between 4.5 and 5.5 mm, preferably 5.1 mm, and an inside diameter of between 4 and 5 mm, preferably 4.5 mm. Additionally, in one embodiment of the present invention, the mid wall section of the body portion of the fixing device has an outside diameter, at its widest point, of between 5.5 and 7 mm, preferably 6.3 mm.

The aforementioned dimensions are preferred sizes for fixing devices for use in femoral bones, for example. The particular dimensions of the fixing device may be selected in accordance with the particular type of bone with which the device is to be used or the relative size of the bone which may depend on age of the bone or the like.

When a screw or the like is tightened within a fixing device located within an aperture in a bone structure, any torque applied to rotate the screw may be imparted to the fixing device by frictional engagement of the thread of the screw or the like with the fixing device, causing rotation thereof. To eliminate, or at least substantially prevent the fixing device from rotating, the outer surface of at least a portion of the fixing device may comprise an abrasive material to increase the frictional coefficient between the fixing device and the inner surface of the bone aperture.

Alternatively, or additionally, the fixing device may be held stationary by some means while a screw or the like is tightened therein. The fixing device may be, for example, adapted to engage a device for preventing rotation of the fixing device. Preferably, the head portion of the fixing device is adapted to engage an applicator device which, in use, prevents rotation of the fixing device.

The head portion of the fixing device may comprise at least one detent for receiving a respective at least one tooth on an applicator device.

Alternatively, the head portion of the fixing device may comprise at least one tooth portion for engaging a respective at least one detent in an applicator device.

According to a second aspect of the present invention there is provided a fixing device for use with a threaded body, said fixing device comprising:
an elongate body portion having a longitudinal bore extending therethrough, said bore adapted for receiving a threaded body;
a head portion having a diameter greater than that of the body portion; and
at least one spiral slot located in the body portion of the fixing device, said at least one spiral slot winding in a clock-wise direction around the body portion; such that, in use, said fixing device is inserted into an aperture in a surface and a threaded body is inserted into the bore and tightened therein by rotation relative to the fixing device, causing axial compression of the fixing device and circumferential expansion of at least a portion thereof to secure the fixing device within the aperture.

Preferably, the fixing device comprises a plurality of spiral slots.

Preferably also, the fixing device is for use in a surface having a wall thickness less than the length of the fixing device, such that when a fixing device is inserted into an aperture in a surface, a portion of the fixing device will protrude beyond the rear face of the surface.

Conveniently, the wall thickness of the body portion of the fixing device varies over the length thereof such that the body portion adjacent the head portion has a thinner wall thickness than that of the remaining body portion. Preferably, the mid wall section of the body portion has an increased wall thickness profile relative to the remainder of the body portion.

Preferably, in use, when a threaded body is tightened within a fixing device located in an aperture in a surface, the axial forces produced will initially collapse and buckle the thin walled portion of the fixture device, causing circumferential expansion and engagement with the inner surface of the aperture. Such initial expansion accommodates for various aperture diameters and surface wall thicknesses, as the thin walled portion allows for significant axial shrinkage of the fixing device.

Conveniently, further tightening of the threaded body will cause circumferential expansion of the mid wall section of the body portion protruding beyond the rear face of the surface, producing a distended mid wall section. At this stage, the distended portion prevents the fixing device from being extracted from the aperture under any significant extraction force.

Preferably, continued tightening of the threaded body will cause further axial compression of the fixing device and will cause the expanded mid section to be compressed against the rear face of the surface, firmly securing the fixing device within the surface aperture.

Preferably, the outer surface of at least a portion of the fixing device may comprise an abrasive material to increase the frictional coefficient between the fixing device and the inner surface of an aperture, to eliminate, or at least substantially prevent, the fixing device from rotating when a screw or the like is rotated therein

Alternatively, or additionally, the fixing device may be held stationary by some means while a screw or the like is tightened therein. The fixing device may be, for example, adapted to engage a device for preventing rotation of the fixing device. Preferably, the head portion of the fixing device is adapted to engage an applicator device which, in use, prevents rotation of the fixing device.

The head portion of the fixing device may comprise at least one detent for receiving a respective at least one tooth on an applicator device.

Alternatively, the head portion of the fixing device may comprise at least one tooth portion for engaging a respective at least one detent in an applicator device.

According to a third aspect of the present invention there is provided an applicator device for preventing rotation of a fixing device when a threaded body is tightened therein, said applicator device comprising an elongate body portion having a throughbore, said elongate body having a first end adapted for engagement with a fixing device such that, in use, said first end will engage a portion of a fixing device and will be retained from rotational motion, and a tool will be inserted through the throughbore to engage and rotate the threaded body, allowing the threaded body to be rotated while the fixing device is held stationary.

Preferably, the throughbore is substantially co-axially aligned with the elongate body of the applicator device.

Preferably, the applicator device comprises a handle portion, which handle portion may be located at a second end of the elongate body, opposite the first end thereof. Preferably, the handle portion has a throughbore aligned co-axially with the throughbore of the elongate body.

Alternatively, the handle portion may be a lever fixed to the elongate body.

Conveniently, the applicator device further comprises gripping means for allowing the applicator device to be gripped and prevented from rotating when a threaded body is rotated within a fixing device. The gripping means may be provided on the outer surface of the elongate body. Alternatively, the gripping means may be provided on the handle portion. Preferably, the gripping means comprises a textured surface such as, for example, a knurled surface.

The applicator device may be integrally provided with a tool for rotating a threaded body. This would provide for the application of a fixing device with the use of a single tool.

Preferably, the device comprises at least one tooth portion extending longitudinally from the first end of the elongate body, said at least one tooth portion for engaging a respective at least one corresponding detent in the fixing device. More preferably, the device comprises two teeth portions.

Alternatively, the device may comprise at least one detent in the first end thereof for receiving a respective at least one tooth portion extending from the fixing device. Preferably, the device comprises two detents.

Preferably, the applicator device according to the fourth aspect of the present invention may be used with the fixing device as defined in the first, second and third aspects.

According to a fourth aspect of the present invention there is provided a method of performing surgery, said method involving the steps of:
providing a fixing device having an elongate body portion and a longitudinal bore extending therethrough, said bore adapted for receiving a threaded body;
inserting said fixing device into an aperture in a bone structure;
aligning an orifice of a device to be fixed to the bone structure with the bore of the fixing device; and
inserting a threaded body through the orifice of the device and into the bore of the fixing device and tightening therein by rotation relative to the fixing device, causing axial compression of the fixing device and circumferential expansion of at least a portion thereof to secure the fixing device within the aperture and to secure the device to the surface of the bone structure.

These and other aspects of the present invention will now be described, by way of example only, with reference to the accompanying drawings, in which:

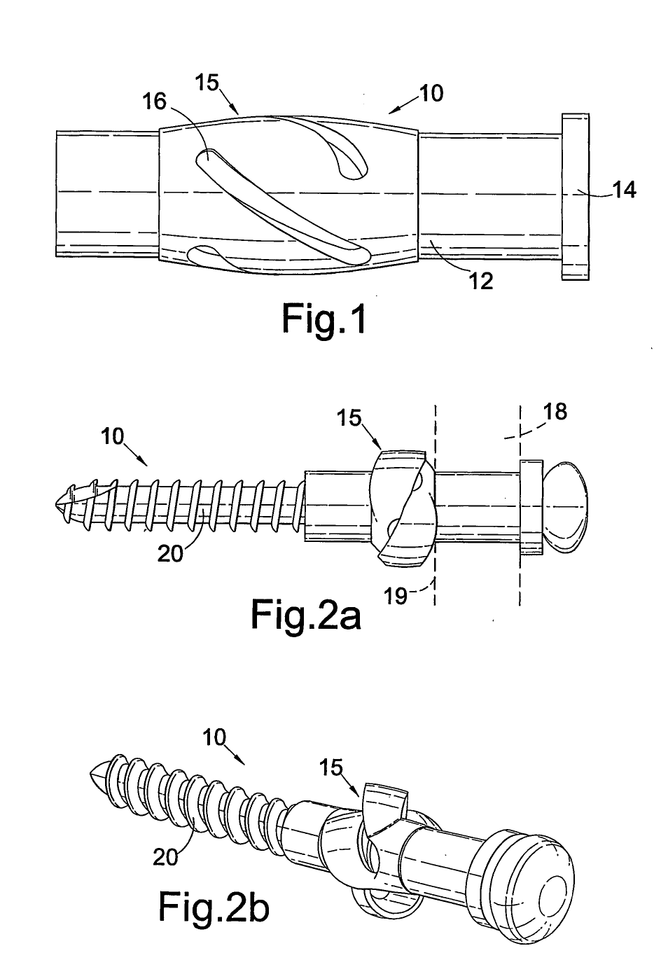

FIG. 1 is a representation of a fixing device in accordance with a preferred embodiment of the present invention;

FIG. 2a is a view of the fixing device of FIG. 1 shown in an axially compressed state;

FIG. 2b is a perspective view of the fixing device of FIG. 2a;

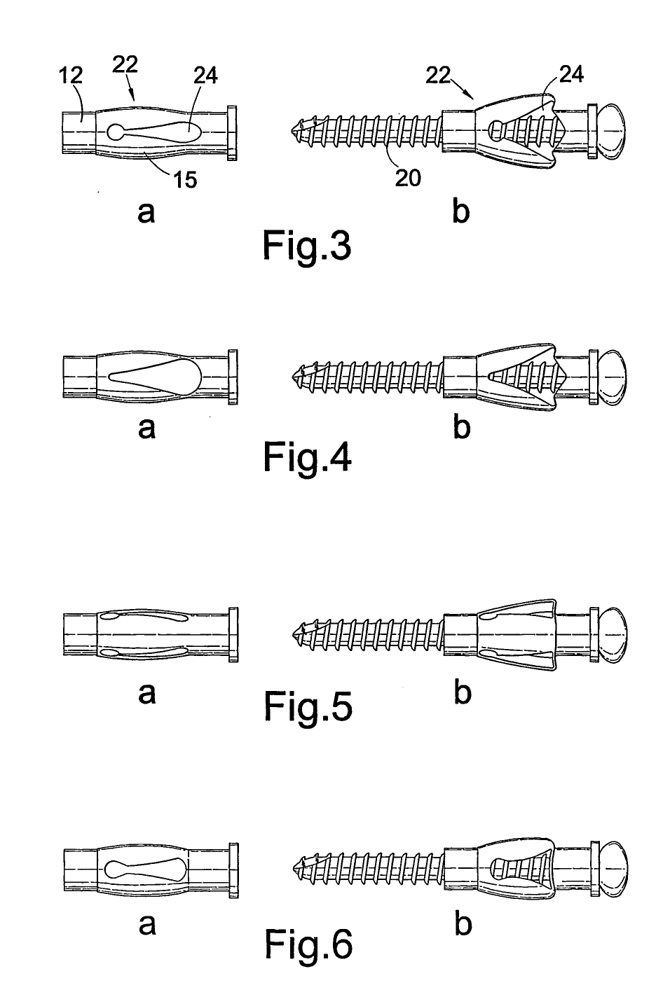

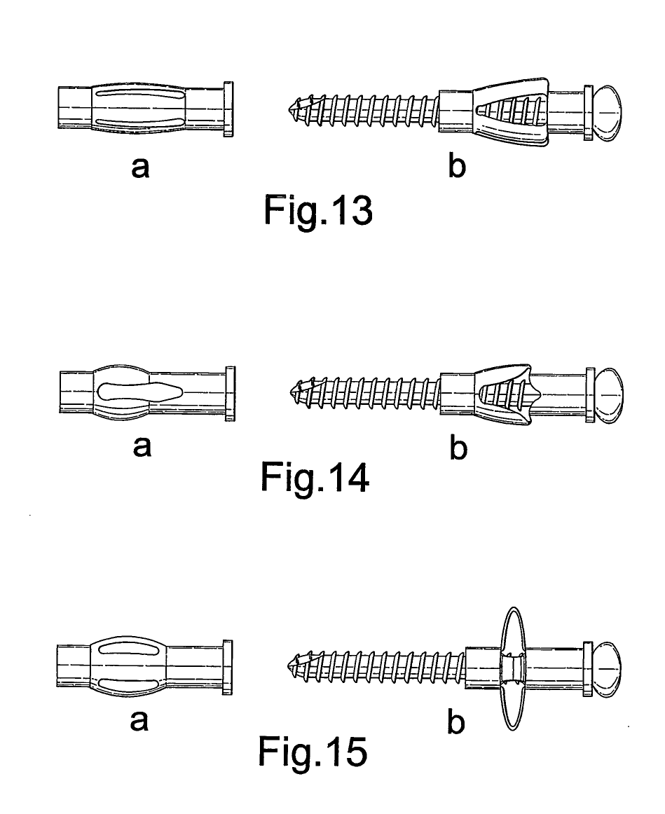

FIGS. 3 to 15 are representations of fixing devices in accordance with alternative embodiments of the present invention;

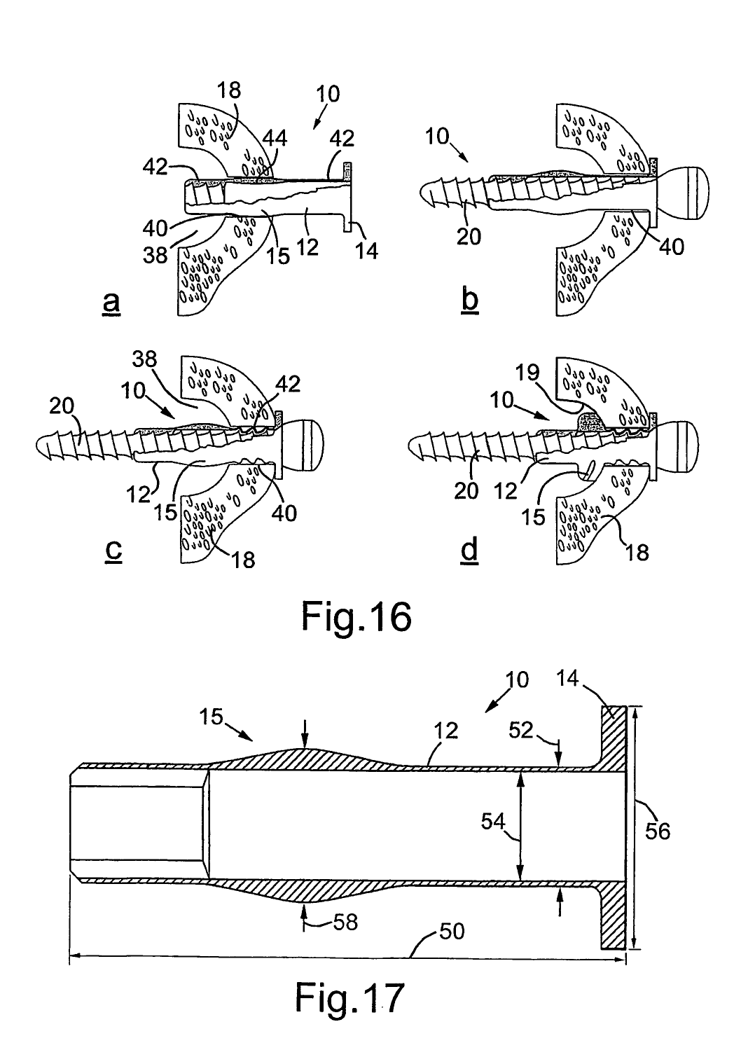

FIG. 16a to 16d show the steps by which a fixing device according to the present invention is secured in an aperture within a bone structure;

FIG. 17 is a cross-sectional view of a fixing device in accordance with a preferred embodiment of the present invention; and

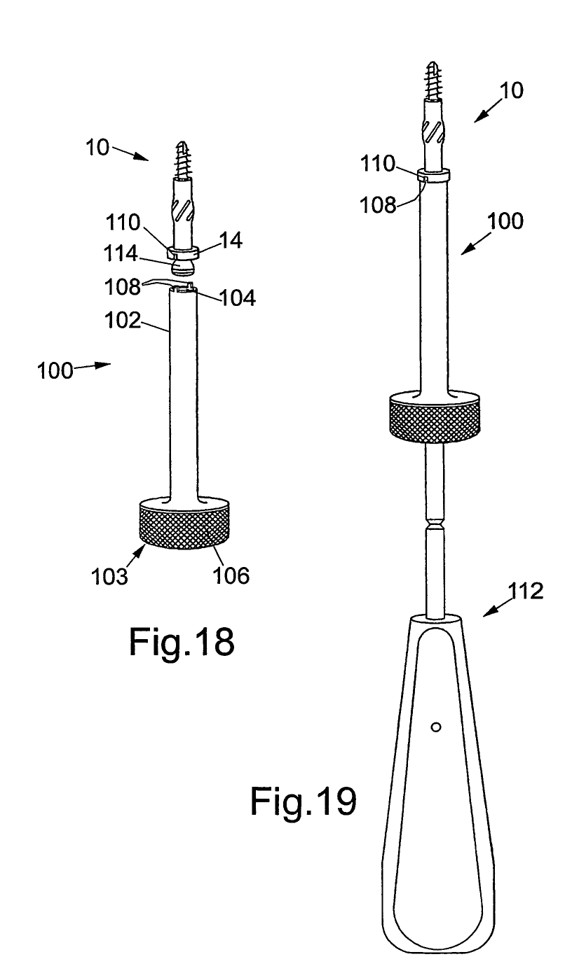

FIGS. 18 and 19 are representations of an applicator device for use with a fixture device in accordance with an aspect of the present invention.

Reference is first made to FIG. 1 in which there is shown a fixing device in accordance with a preferred embodiment of the present invention. The fixing device, generally indicated by reference numeral 10, comprises an elongate body portion 12 having a longitudinal bore (not shown) extending therethrough, a head portion 14 having a diameter greater than that of the body portion 12, and a number of spiral slots 16 located in the body portion 12 at its mid section 15, which spiral slots 16 wind in a clockwise direction. In the embodiment shown, four spiral slots 16 are provided which each extend approximately 90° around the circumference of the elongate body portion 12.

Reference is now made to FIGS. 2a and 2b in which there is shown the fixing device 10 of FIG. 1 located within a bone structure 18 (shown in broken outline in FIG. 2a). The fixing device is located in an aperture in the bone structure 18 and a screw 20 has been inserted and tightened within the bore of the fixing device 10. The action of tightening the screw 20 has axially compressed the fixing device 10 causing circumferential expansion of the mid section 15 thereof, thus securing the fixing device 10 within the bone structure 18. Upon tightening the screw 20, the slots 16 are closed which provides a solid structure on the rear face 19 of the bone structure 18.

Reference is now made to FIGS. 3 to 15 in which there is shown alternative embodiments of the fixing device in initial (Figures a) and axially compressed (Figures b) states. The Fixing device 22 of FIG. 3a comprises tear shaped apertures 24, located in the mid section 15 of the body portion 12. When a screw 20 is tightened within the fixing device 22, the fixing device 22 is axially compressed and the mid section 15 of the body portion 12 is expanded circumferentially. Upon circumferential expansion, the tear shaped slots 24 are widened. The fact that the slots 24 are capable of widening allows is significant expansion of the mid portion 15 of the fixing device 22. Alternative tear shaped slot arrangements are shown in FIGS. 4 to 6.

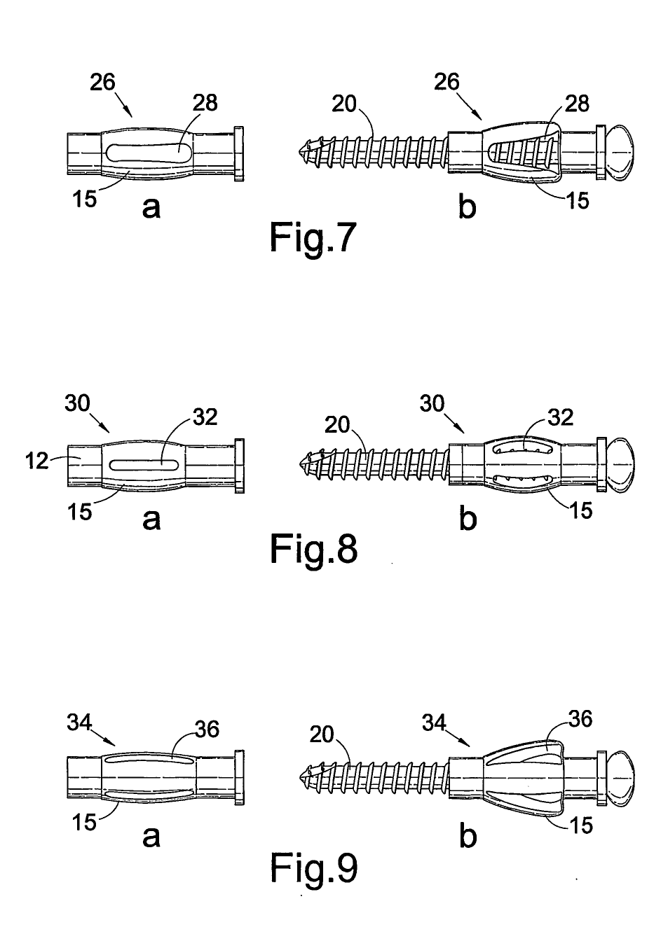

In an alternative embodiment, shown in FIG. 7a, a fixing device 26 comprises longitudinally extending wide slots 28 aligned parallel to the longitudinal axis of the fixing device 26. As shown in FIG. 7b, when a screw 20 is tightened within the fixing device 26, the slots 28 are widened as the mid section 15 of the fixing device 26 expands.

Referring now to FIG. 8a, a further embodiment of the present invention will now be described. A fixing device 30 comprises a number of short longitudinally extending slots 32 disposed around the mid section 15 of a body portion 12 of the fixing device 30. As shown in FIG. 8b, the slots 32 have slightly widened due to the tightening of a screw 20 within the fixing device 30, causing circumferential expansion thereof.

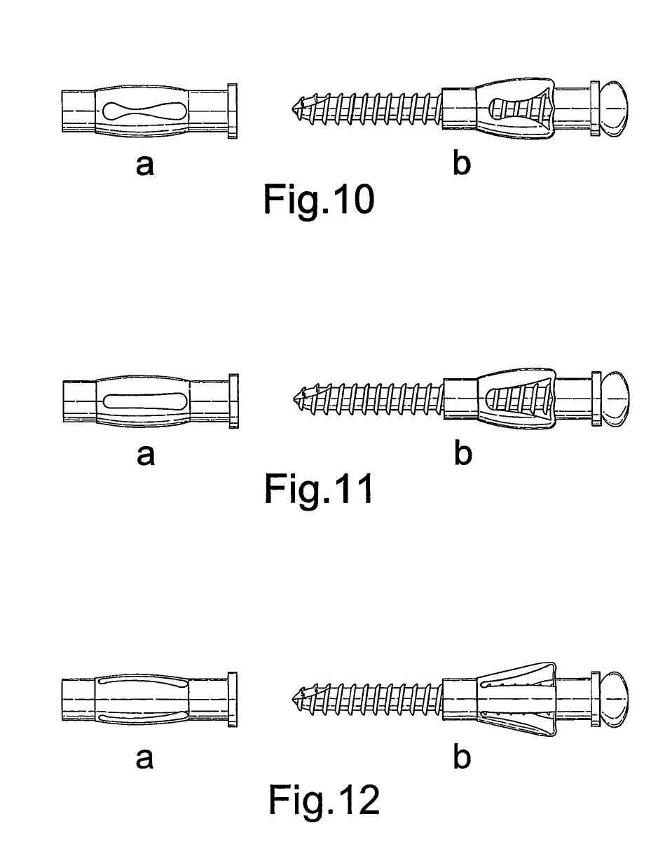

A still further alternative embodiment of the present invention is shown in FIG. 9a. A fixing device 34 comprises longitudinally extending slots 36, similar to those slots 28, 32 of the fixing devices 26, 30 of FIGS. 7 and 8. The slots 36 in this embodiment, however, are substantially thinner, and in particular longer than those of the device 30 shown in FIG. 8. Upon tightening a screw 20 within the fixing device 34, as shown in FIG. 9b, the slots 36 widen as the mid section 15 of the fixing device 34 expands. Alternative embodiments having longitudinal slots are shown in FIGS. 10 to 15.

The process by which a fixing device according to the present invention is secured in an aperture will now be described in more detail with reference to FIGS. 16a to 16d. Referring initially to FIG. 16a, a fixing device 10, shown in partial cross-section, is inserted through an aperture 40 in a bone structure 18 and into the medullary cavity 38. As is evident from FIG. 16a, the wall thickness of the body portion 12 of the fixing device 10 varies over the length thereof. The body portion 12 comprises a thin walled section 42 positioned adjacent the head portion 14 of the fixing device 10. The mid section 15 has an increased wall thickness 44, and the end of the body portion 12, opposite the head portion 14, has a wall portion 46, having a thickness greater than that of the thin walled portion 42.

Once fully inserted into the aperture 40, as shown in FIG. 16b, a screw 20 is fully inserted into the bore of the fixing device, normally by rotating therein. Further rotation, and thus tightening of the screw 20 within the fixing device 10 will produce axial compressive forces which will initially collapse and buckle the thin walled portion 42 of the body portion 12, as shown in FIG. 16c. Such initial buckling accommodates for various aperture 40 diameters and cortical wall thicknesses of the bone structure 18, as the thin walled portion 42 allows for significant axial shrinkage of the fixing device 10.

Further tightening of the screw 20 will cause circumferential expansion of the mid section 15 located within the medullary cavity 38. At this stage, the increased thickness wall portion 44 is distended and prevents the fixing device 10 from being extracted from the aperture under any significant extraction force.

Continued tightening of the screw, as shown in FIG. 16d, will cause further axial compression of the fixing device 10 and will cause the previously expanded mid portion 15 of the body portion 12 to be compressed against the inner face 19 of the cortical wall of the bone structure 18, firmly securing the fixing device 10 within the bone structure 18.

Referring to FIG. 17, the relative dimensions of a fixing device in accordance with one embodiment of the present invention will now be described. The fixing device 10 shown in FIG. 17 is for use in femoral bones and has an overall length 50 of 23 mm, an outer body wall 12 diameter 52 of 5.1 mm, an inner diameter 54 of 4.5 mm and a head portion 14 diameter 56 of 10 mm. The mid wall section 15 of the body portion 12 of the fixing device 10 has an outside diameter 58, at its widest point, of 6.3 mm.

Reference is now made to FIGS. 18 and 19 in which there is shown an applicator device for use with the fixing plug of the present invention. The applicator device, generally indicated by reference numeral 100, is used to prevent a fixing device from rotating when a threaded body or the like is tightened therein. The applicator device 100 comprises an elongate body portion 102, a handle portion 103 and a bore 104 extending through the elongate body portion 102 and the handle portion 103. As shown in FIG. 18, the handle portion 103 comprises a knurled grip 106 to allow said handle 103 to be firmly held when in use.

The applicator device 100 further comprises two teeth portions 108 for engaging respective detents 110 (only one shown) in the head portion 14 of a fixture device 10.

The use of the applicator 100 will now be described, with reference to FIG. 19 of the drawings. The teeth portions 108 of the applicator 100 are inserted into the detents 110 of the fixing device 10, and the applicator 100 is held to prevent rotation of the fixing device 10. A portion of a tool 112 is inserted into the throughbore 104 of the applicator 100 to engage and rotate a screw 20 within the fixing device 10. Thus, as the tool 112 is rotated to tighten the screw 20, the fixing device 10 will be prevented from rotation.

It would be obvious to a person of skill in the art that the embodiments hereinbefore described are merely exemplary of the present invention and that various modifications may be made thereto without departing from the scope of the present invention. For example, the fixing device may be used for providing strong fixing means in any surface such as plaster board, for example. The fixing device may have any suitable slot formation which would allow the body portion of the device to sufficiently expand to provide proper fixation. The body portion of the fixing device may be provided with an abrasive material to increase the frictional coefficient between the device and the aperture into which the device is inserted, to substantially prevent rotation of the device when a screw or the like is tightened therein.

The particular dimensions of the fixing device may be selected in accordance with the particular type of bone with which the device is to be used or the relative size of the bone which may depend on age of the bone, for example.

The applicator device may comprise a lever type handle located adjacent the elongate body portion. Additionally, the applicator device may comprise detents for receiving teeth which may extend from the head portion of a fixing device. In general, any means for engaging the head portion of the fixing device while inserted in an aperture may be utilised to prevent the device from rotating.

1. A fixing device for use in orthopaedics, said fixing device comprising an elongate body portion having a longitudinal bore extending therethrough, said bore adapted for receiving a threaded body, wherein a mid wall section of the body portion has an increased wall thickness profile relative to a remainder of the body portion, said mid wall section defining at least one spiral aperture therein, such that, in use, said fixing device is inserted into an aperture in a bone structure and a threaded body is inserted into the bore and tightened therein by rotation relative to the fixing device, resulting in axial compression of the fixing device to close the at least one spiral aperture and cause circumferential expansion of the mid wall section of the fixing device to secure the fixing device within the aperture of the bone structure.

2. A fixing device as claimed in claim 1, wherein the fixing device further comprises a head portion having a diameter greater than that of the body portion and selected to be greater than that of the bone aperture into which the device is to be inserted.

3. A fixing device as claimed in claim 1, wherein the device is of a sufficient length so as to extend through the cortical wall of a bone and into its intramedullary cavity.

4. A fixing device as claimed in claim 3, wherein a portion of the fixing device which extends into the cavity is expandable to form a load bearing surface on an inner surface of the cortical wall.

5. A fixing device as claimed in claim 1, wherein the fixing device has a wall thickness which varies over the length thereof.

6. A fixing device as claimed in claim 2, wherein a section of the body portion adjacent the head portion has a thinner wall thickness than that of the remaining body portion.

7. A fixing device as claimed in claim 6, wherein, in use, when a threaded body is tightened within a fixing device located in a bone aperture, the axial forces produced initially collapse and buckle the body portion section, causing circumferential expansion and engagement with the an inner surface of the aperture.

8. A fixing device as claimed in claim 7, wherein, in use, further tightening of the threaded body causes circumferential expansion of the mid wall section of the body portion producing a distended mid wall section.

9. A fixing device as claimed in claim 7, wherein, in use, continued tightening of the threaded body causes further axial compression of the fixing device and causes the expanded mid section to be compressed against an inner surface of the bone in which the fixing device is inserted.

10. A fixing device as claimed in claim 1, wherein the fixing device further comprises a plurality of spiral apertures located in the mid section of the body portion.

11. A fixing device as claimed in claim 1, wherein the at least one spiral aperture winds in a clockwise direction

12. A fixing device as claimed in claim 1, wherein the at least one spiral aperture winds in an anti-clockwise direction.

13. A fixing device as claimed in claim 1, wherein when a threaded body is tightened within the longitudinal bore and the at least one aperture is closed, a solid structure is provided on the inner surface of the bone in which the fixing device is inserted.

14. A fixing device as claimed in claim 1, wherein the at least one spiral aperture extends at least 45° around a circumference of the fixing device.

15. A fixing device as claimed in claim 1, wherein the at least one spiral aperture extends less than 360° around a circumference of the fixing device.

16. A fixing device as claimed in claim 1, wherein the fixing device is manufactured from a synthetic fibre material.

17. A fixing device as claimed in claim 1, wherein the fixing device is made from a plastics material.

18. A fixing device as claimed in claim 1, wherein an outer surface of at least a portion of the fixing device comprises an abrasive material to increase a frictional coefficient between the fixing device and an inner surface of the bone aperture.

19. A fixing device as claimed in claim 1, wherein the fixing device is held stationary while said threaded body is tightened therein.

20. A fixing device as claimed in claim 19, wherein the fixing device is adapted to engage an applicator device for preventing rotation of the fixing device.

21. A fixing device for use with a threaded body, said fixing device comprising:
an elongate body portion having a longitudinal bore extending therethrough, said bore adapted for receiving a threaded body, wherein a mid wall section of the body portion has an increased wall thickness profile relative to a remainder of the body portion;
a head portion having a diameter greater than that of the body portion; and
at least one spiral slot located in the mid wall section of the body portion of the fixing device, said at least one spiral slot winding in a clock-wise direction around the body portion; such that, in use, said fixing device is inserted into an aperture in a surface of an object and a threaded body is inserted into the bore and tightened therein by rotation relative to the fixing device, causing axial compression of the fixing device and circumferential expansion of at least a portion thereof to secure the fixing device within the aperture.

22. A fixing device as claimed in claim 21, wherein the fixing device further comprises a plurality of spiral slots.

23. A fixing device as claimed in claim 21, wherein the fixing device is for use in orthopaedics.

24. An applicator device for preventing rotation of a fixing device of the type comprising an elongate body portion having a longitudinal bore extending therethrough, said bore adapted for receiving a threaded body, wherein a mid wall section of the body portion has an increased wall thickness profile relative to a remainder of the body portion, said mid wall section defining at least one spiral aperture therein when a threaded body is tightened therein, said applicator device comprising an elongate body having a throughbore, said elongate body having a first end adapted for engagement with a fixing device such that, in use, said first end will engage a portion of said fixing device and will be retained from rotational motion, and a tool will be inserted through the throughbore to engage and rotate the threaded body, allowing the threaded body to be rotated while the fixing device is held stationary.

25. An applicator device for preventing rotation of a fixing device when a threaded body is tightened therein, said applicator device comprising an elongate body having a throughbore, said elongate body having a first end adapted for engagement with a fixing device such that, in use, said first end will engage a portion of said fixing device and will be retained from rotational motion, and a tool will be inserted through the throughbore to engage and rotate the threaded body, allowing the threaded body to be rotated while the fixing device is held stationary.

26. An applicator device as claimed in claim 25, wherein the throughbore is substantially co-axially aligned with the elongate body of the applicator device.

27. An applicator device as claimed in claim 25, wherein the applicator device further comprises a handle portion, which handle portion is located at a second end of the elongate body, opposite the first end thereof.

28. An applicator device as claimed in claim 27, wherein the handle portion has a throughbore aligned co-axially with the throughbore of the elongate body.

29. An applicator device as claimed in claim 27, wherein the handle portion is a lever fixed to the elongate body.

30. An applicator device as claimed in claim 25, wherein the applicator device further comprises gripping means for allowing the applicator device to be gripped and prevented from rotating when said threaded body is rotated within said fixing device.

31. An applicator device as claimed in claim 30, wherein the gripping means is disposed on an outer surface of the elongate body.

32. An applicator device as claimed in claim 30, wherein the gripping means is disposed on a handle portion located at a second, opposite end of the elongate body.

33. An applicator device as claimed in claim 25, wherein the applicator device is integrally provided with a tool for roatating the threaded body.

34. An applicator device as claimed in claim 25, wherein the device further comprises at least one tooth portion extending longitudinally from the first end of the elongate body, said at least one tooth portion for engaging a respective at least one corresponding detent in the fixing device.

35. An applicator device as claimed in claim 25, wherein the device further comprises at least one detent in the first end thereof for receiving a respective at least one tooth portion extending from the fixing device.

36. A method of performing surgery, said method involving the steps of:
providing a fixing device having an elongate body portion and a longitudinal bore extending therethrough, said bore adapted for receiving a threaded body;
inserting said fixing device into an aperture in a bone structure;
aligning an orifice of a device to be fixed to the bone structure with the bore of the fixing device; and
inserting a threaded body through the orifice of the device and into the bore of the fixing device and tightening therein by rotation relative to the fixing device, causing axial compression of the fixing device and circumferential expansion of at least a portion thereof to secure the fixing device within the aperture and to secure the device to a surface of the bone structure.
